(19) United States
(12) Patent Application Publication
(10) Pub. No.: US 2010/0121120 A1
(43) Pub. Date: May 13, 2010

(54) PROCESS FOR UPGRADING HYDROCARBONS

(75) Inventors: Bruce B. Randolph, Bartlesville, OK (US); Bruce Welch, Greenbrier, AR (US); Roland Schmidt, Bartlesville, OK (US); Edward L. Sughrue, II, Bartlesville, OK (US)

Correspondence Address: ConocoPhillips Company - IP Services Group;Attention: DOCKETING, 600 N. Dairy Ashford, Bldg. MA-1135, Houston, TX, 77079, US

(73) Assignee: ConocoPhillips Company, Houston, TX (US)

(21) Appl. No.: 12/607,809

(22) Filed: Oct. 28, 2009

Related U.S. Application Data
(60) Provisional application No. 61/109,700, filed on Oct. 30, 2008.

(51) Int. Cl. C07C 2/00; C07C 11/02

(52) U.S. Cl. 585/16; 585/324

(57) ABSTRACT

A process for upgrading hydrocarbons comprising removal of C5 hydrocarbons from a feedstock, metathesizing said C5 hydrocarbons to C6+ and C4− hydrocarbons, and upgrading said C4− hydrocarbons is disclosed.

### CROSS-REFERENCE TO RELATED APPLICATION

This application claims benefit of U.S. provisional patent application No. 61/109,700 filed Oct. 30, 2008.

### FIELD OF THE INVENTION

The present invention generally related to a process for upgrading hydrocarbons. More particularly, the invention relates to an improved process to provide a gasoline product with a good drivability index and a low Reid Vapor Pressure.

### BACKGROUND OF THE INVENTION

Gasoline regulations limit the amount of sulfur that can be present in motor fuel.

One area of interest from automakers is the distillation index or drivability index (DI), which is a measure of gasoline tendency to vaporize. It is calculated from a gasoline's distillation profile. The specific formula for Drivability Index (DI) is DI(° F.)=1.5(T10)+3(T50)+T90. The variables T10, T50, and T90 are the temperatures (in degrees Fahrenheit) at which 10%, 50% and 90% of the fuel vaporizes, respectively, during a standard ASTM D86 distillation test. To have desirable emissions characteristics, it is preferred that the drivability index is below 1200° F.

Another area of interest from automakers is the Reid Vapor Pressure, which defined as the absolute vapor pressure of volatile crude oil and volatile non-viscous petroleum liquids. A lower Reid Vapor Pressure is desirable.

However, it is challenging to produce gasoline with both the desirable Reid Vapor Pressure and the desirable Drivability Index since Reid Vapor Pressure and Drivability Index tend to act in an opposite fashion in such that Reid Vapor Pressure decreases with an increase in T10 while DI increases with an increase in T10. For example, removal of the lighter fuel components such as nC4 and C5's will shift the T10 and T50 to higher values, resulting in an increase in the Drivability Index unless steps are taken to remove the heavier portion of the gasoline which may result in a significant lost in octane.

Therefore, a hydrocarbon upgrading process that can address the Reid Vapor Pressure and Drivability Index issues simultaneously would be a benefit to both the art and to the economy.

### SUMMARY OF THE INVENTION

One aspect of the invention discloses a process for upgrading hydrocarbons.

One embodiment according to the current invention comprising the following steps:

a) The hydrocarbon feedstock is passed to a first separation zone, where a first hydrocarbon stream and a remaining hydrocarbon stream are separated from the hydrocarbon feedstock. The first hydrocarbon stream comprises compounds having 5 carbon atoms per molecule (C5);

b) This first hydrocarbon stream is then passed to a metathesis reaction zone, where the first hydrocarbon stream undergoes a metathesis reaction to form metathesis reaction product stream comprising compounds having less than five carbon atoms per molecule (C4−), compounds having five carbon atoms per molecule (C5), and compounds having at least six carbon atoms per molecule (C6+);

c) The metathesis reaction product stream comprising C₄₋, C₅ and C₆₊ hydrocarbons is then passed to a second separation zone. There, the metathesis reaction product stream is separated into a second hydrocarbon stream comprising compounds having less than 6 carbon atoms per molecule (C5−) and into a third hydrocarbon stream comprising compounds having at least 6 carbon atoms per molecule (C6+);

d) The second hydrocarbon stream is then passed to a third separation zone. There, the second hydrocarbon stream is separated to form a fourth hydrocarbon stream comprising compounds having less than 5 carbon atoms per molecule (C4−) and a fifth hydrocarbon stream comprising compounds having 5 carbon atoms per molecule (C5).

e) The fourth hydrocarbon stream is passed to a hydrocarbon upgrading zone.

Another embodiment according to the current invention further comprises steps such as i) passing the third hydrocarbon stream to a gasoline blending zone; ii) recycling the fifth hydrocarbon stream to the metathesis reaction zone; iii) passing the remaining hydrocarbon stream in first separation zone to and gasoline blending zone; or any combination thereof.

The hydrocarbon feedstock according to one embodiment of the current invention may comprise compounds with 2 to 20 carbon atoms per molecule.

The hydrocarbon feedstock according to one embodiment of the current invention may contain less than 300 ppmv dienes, or less than 100 ppmv dienes. Within dienes also means diolefins.

The hydrocarbon feedstock according to one embodiment of the current invention may contain less than 30 ppmv sulfur, or less than 10 ppmv sulfur, or less than 5 ppmv sulfur.

The upgrading zone according to one embodiment of the current invention may be an alkylation reaction zone or an oligomerization reaction zone.

The temperature in the metathesis reaction zone according to one embodiment of the current invention may be in the range of from about 700° F. to about 800° F.

The metathesis catalyst according to one embodiment of the current invention may be silica-supported tungsten oxide in conjunction with magnesium oxide.

The metathesis catalyst according to one embodiment of the current invention may be regenerated with hydrogen.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 is a schematic flow diagram presenting one embodiment of the present invention.

### DETAILED DESCRIPTION OF THE PREFERRED EMBODIMENTS

In accordance with the present invention, a process is provided for upgrading hydrocarbon feedstock. The process involves separating C5 compound from the hydrocarbon feedstock; metathezing C5 compound to produce C4−, C5, and C6+ compounds; separating C5 and C6+ compounds; upgrading C4− compounds; and recycling C5 for metathesis.

The process described herein is an integrated process. It refers to a process which involves a sequence of steps, some of which may be parallel to other steps in the process, but which are interrelated or dependent upon either to earlier or late steps in the overall process.

Any suitable hydrocarbon feedstock can be utilized in the present inventive process. Suitable hydrocarbon feedstock may comprise, but not limited to, the compounds with 2 to 20 carbon atoms per molecule. Suitable hydrocarbon feed stock may also contain, but not limited to, less than 300 ppmv dients, or less than 100 ppmv dients. Suitable hydrocarbon feed stock may further contain, but not limited to, less than 30 ppmv sulfur, or less than 10 ppmv sulfur, or less than 5 ppmv sulfur.

The hydrocarbon feedstock is passed to a first separation zone, where first hydrocarbon comprising compounds having 5 carbon atoms per molecule and a remaining hydrocarbon stream are separated from the hydrocarbon feedstock

While the remaining hydrocarbon stream is passed to a gasoline blending zone, the first hydrocarbon stream is passed to a metathesis reaction zone, where the first hydrocarbon stream undergoes a metathesis reaction. “Metathesis” refers to the interchange of carbon atoms between a pair of double bonds which is catalyzed by various metal compounds. In the present invention, the first hydrocarbon stream, which is passed into the metathesis reaction zone, is comprised of compounds having 5 carbon atoms per molecule, and the metathesis reaction product stream is comprised of olefins having either 4, 5, or 6 carbon atoms per molecule.

Any suitable metathesis catalyst can be utilized in the metathesis reaction zone. Suitable catalysts include, but are not limited to, transition metal halides or oxides with an alkylating co-catalyst, titanocene-based catalysts, ruthenium catalysts supported by phosphine ligands, and tungsten and/or molybdenum-containing catalysts. Other suitable catalysts are described, for example, in U.S. Pat. Nos. 4,522,936 and 4,071,471, the contents of which are incorporated herein by reference. The catalyst according to an embodiment of the current invention is silica-supported tungsten oxide in conjunction with magnesium oxide. The catalyst according to an embodiment of the current invention may be regenerated by the use of hydrogen.

The temperature in the metathesis reaction zone depends on the type of catalyst used. For one embodiment where a tungsten oxide/magnesium oxide catalyst is used, the temperature in the metathesis reaction zone will be within the range of from about 700° F. to about 800° F.

The metathesis reaction product stream comprising C₄, C₅ and C₆ olefins is then passed to a second separation zone. There, the metathesis reaction product stream is then separated into a second hydrocarbon stream comprising compounds having less than 6 carbon atoms per molecule and into a third hydrocarbon stream comprising compounds having at least 6 carbon atoms per molecule.

The second hydrocarbon stream is then passed to a third separation zone. There, the second hydrocarbon stream is separated to form a fourth hydrocarbon stream comprising compounds having less than 5 carbon atoms per molecule and a fifth hydrocarbon stream comprising compounds having 5 carbon atoms per molecule.

With the third hydrocarbon stream being passed to a gasoline blending zone and the fifth hydrocarbon stream being recycled back to the metathesis reaction zone for metathesis reaction as described above, the fourth hydrocarbon stream is passed to a hydrocarbon upgrading zone where the C4− compounds undergoes a hydrocarbon upgrading process.

The hydrocarbon upgrading zone according to one embodiment of the present invention may be an alkylation reaction zone, where the C4− compounds undergoes an alkylation reaction. Suitable alkylation reaction unit, condition and catalysts used therefore, are described, for example, in U.S. Pat. Nos. 6,395,945 and 5,254,790, the contents of which are incorporated herein by reference.

The hydrocarbon upgrading zone may also be an oligomerization reaction zone, where the C4− compounds undergoes an oligomerization reaction and produces higher octane low RVP gasoline blend.

Any suitable separation method may be used in any of the separation zones of the present invention mentioned above, suitable method may be, but not limited to, fractional distillation.

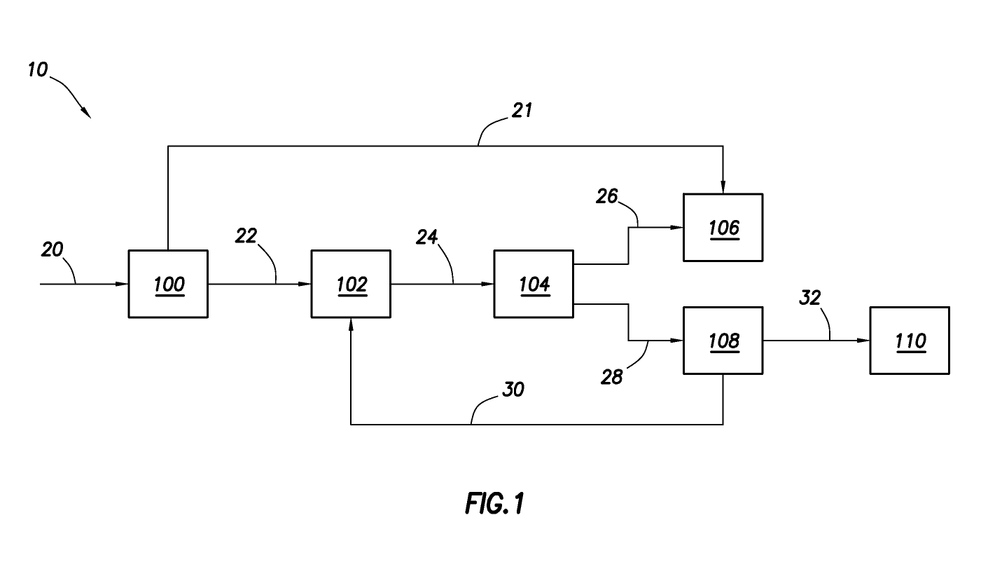

Now referring to FIG. 1, a process system 10 is depicted which comprises the following steps.

A hydrocarbon feedstock is passed to a first separation zone 100 via conduit 20. The feedstock is separated into first hydrocarbon stream comprising compounds having 5 carbon atoms per molecule and a remaining hydrocarbon stream without C5 components. The remaining hydrocarbon stream without the C₅ components passes to gasoline blending zone 106 via conduit 21. The first hydrocarbon stream then passes into metathesis reaction zone 102 via conduit 22 to form a metathesis reaction product stream which passes into a second separation zone 104 via conduit 24. In second separation zone 104, the metathesis reaction product stream is separated into a second hydrocarbon stream and a third hydrocarbon stream. The third hydrocarbon stream comprises compounds having at least six carbon atoms per molecule and it passes through conduit 26 to gasoline blending zone 106. The second hydrocarbon stream comprises compounds having 5 or less carbon atoms per molecule. It passes through conduit 28 to third separation zone 108. There, the second hydrocarbon stream is separated into a fourth hydrocarbon stream comprising compounds having less than 5 carbon atoms per molecule and a fifth hydrocarbon product stream comprising compounds having 5 carbon atoms per molecule. The fifth hydrocarbon product stream returns to metathesis reaction zone 102 via conduit 30. The fourth hydrocarbon product stream passes via conduit 32 to hydrocarbon upgrading zone 110.

The following examples are presented to further illustrate this invention and are not to be construed as unduly limiting the invention as set out in the specification and the appended claims.

### EXAMPLE I

A 5.33-gram quantity of an MgO/WO₃/SiO₂ metathesis catalyst was contacted with a feed comprising the components listed below in Table I at a feed rate of 40 ml/hr. The weight hourly space velocity (WHSV) was 4.6 hr⁻¹ and the liquid hourly space velocity (LHSV) was 3.6 hr⁻¹. The temperature set point was 700° F. Results (on wt % basis) were measured hourly and are shown in Table I.

TABLE I

| Catalyst: | MgO/WO₃/SiO₂ Metathesis Catalyst | | | |
|---|---|---|---|---|
| Catalyst Weight, g | 5.33 | 11 cc catalyst volume | | |
| WHSV (hr⁻¹) | 4.8 | 1.17 olefin only | | |
| Feed Rate (mL/hr) | 40 | 24.71 g/hr feed | | |
| LHSV (hr⁻¹) | 3.6 | | | |
| Temp Set Pt, ° F. | | 700 | 700 | 700 |
| Component | Feed #1 | Prod 1 | Prod 2 | Prod 3 |
| Ethylene | | 0.065 | 0.071 | 0.028 |
| Propane | 0.000 | 0.000 | 0.000 | 0.000 |
| Propylene | 0.008 | 1.238 | 1.180 | 0.599 |
| Isobutane | 0.078 | 0.097 | 0.080 | 0.075 |
| Isobutene | 0.533 | 2.088 | 1.953 | 1.257 |
| Normal Butane | 0.571 | 0.561 | 0.564 | 0.554 |
| 2-butene trans | 0.384 | 1.425 | 1.417 | 0.966 |
| 2-butene cis | 0.304 | 0.959 | 1.009 | 0.694 |
| 3-methyl butene-1 | 1.258 | 0.487 | 0.511 | 0.639 |
| Isopentane | 48.171 | 49.000 | 49.082 | 48.697 |
| Isopentene | 3.204 | 1.059 | 1.195 | 1.732 |
| 2-methyl butene-1 | 8.523 | 3.639 | 3.831 | 4.435 |
| Normal Pentane | 13.220 | 13.577 | 13.386 | 13.259 |
| Trans-2-pentene | 9.619 | 4.270 | 5.207 | 6.995 |
| Cis-2-pentene | 4.502 | 2.167 | 2.557 | 3.419 |
| 2-methyl butene-2 | 8.552 | 9.029 | 9.772 | 11.353 |
| Unknown C₁-C₅ | 0.187 | 0.001 | 0.001 | 0.001 |
| C₆⁺ | 0.000 | 10.403 | 8.255 | 5.325 |
| Total | 99.114 | 100.000 | 100.000 | 100.000 |
| Total C5 = Conv | | 42.086 | 35.294 | 19.869 |
| C4 = Selectivity | | 21.663 | 25.093 | 23.938 |
| C6 + Selectivity | | 69.320 | 65.592 | 75.160 |

### EXAMPLE II

The catalyst in Example I was then purged overnight with nitrogen at a rate of 50 sccm. The metathesis reaction was then run again with the same conditions as Example I, except that the temperature set point was 760° F. The results (on wt % basis) were once again measured and are shown in Table II.

TABLE II

| | Temp. Set Pt, ° F. | | | | |
|---|---|---|---|---|---|
| | 760 | 760 | 760 | 760 | 760 |
| | Prod 4 | Prod 5 | Prod 6 | Prod 7 | Prod 8 |
| Ethylene | 0.092 | 0.075 | 0.100 | 0.073 | 0.066 |
| Propane | 0.000 | 0.000 | 0.000 | 0.000 | 0.000 |
| Propylene | 1.579 | 1.271 | 1.514 | 1.201 | 1.108 |
| Isobutane | 0.077 | 0.075 | 0.076 | 0.075 | 0.075 |
| Isobutene | 2.212 | 1.958 | 2.225 | 1.861 | 1.740 |
| Normal Butane | 0.553 | 0.555 | 0.558 | 0.552 | 0.552 |
| 2-butene trans | 1.635 | 1.466 | 1.635 | 1.409 | 1.341 |
| 2-butene cis | 1.193 | 1.073 | 1.193 | 1.034 | 0.986 |
| 3-methyl butene-1 | 0.508 | 0.572 | 0.495 | 0.589 | 0.628 |
| Isopentane | 47.888 | 48.778 | 48.718 | 48.713 | 48.697 |
| Isopentene | 0.874 | 1.109 | 0.899 | 1.176 | 1.273 |
| 2-methyl butene-1 | 3.781 | 4.156 | 3.944 | 4.228 | 4.325 |
| Normal Pentane | 13.099 | 13.359 | 13.318 | 13.353 | 13.352 |
| Trans-2-pentene | 3.776 | 4.615 | 4.023 | 4.806 | 5.047 |
| Cis-2-pentene | 1.905 | 2.322 | 2.029 | 2.422 | 2.552 |
| 2-methyl butene-2 | 9.178 | 9.953 | 9.451 | 10.164 | 10.419 |
| Unknown C₁-C₅ | 0.002 | 0.003 | 0.005 | 0.006 | 0.004 |
| C₆⁺ | 11.740 | 8.734 | 9.917 | 8.413 | 7.902 |
| Total | 100.000 | 100.000 | 100.000 | 100.000 | 100.000 |
| Total C5 = Conv | 43.850 | 36.264 | 41.553 | 34.419 | 32.010 |
| C4 = Selectivity | 24.419 | 25.334 | 25.863 | 25.119 | 24.931 |
| C6 + Selectivity | 75.083 | 67.540 | 66.928 | 68.546 | 69.233 |

### EXAMPLE III

The catalyst was then regenerated with a nitrogen/hydrogen combination flow at a rate of 50 sccm for one hour. This was followed by a 50 sccm nitrogen purge overnight. The metathesis reaction was run, with the reaction conditions the same as in Example II. The results (on wt % basis) are shown in Table III.

TABLE III

Temp Set Pt, ° F.

| | 760 | 760 | 760 | 760 |
|---|---|---|---|---|
| | Prod 9 | Prod 10 | Prod 11 | Prod 12 |
| Ethylene | 0.085 | 0.089 | 0.015 | 0.045 |
| Propane | 0.000 | 0.000 | 0.000 | 0.000 |
| Propylene | 1.503 | 1.191 | 0.313 | 0.782 |
| Isobutane | 0.075 | 0.081 | 0.072 | 0.074 |
| Isobutene | 2.202 | 1.946 | 0.905 | 1.398 |
| Normal Butane | 0.553 | 0.557 | 0.542 | 0.550 |
| 2-butene trans | 1.668 | 1.347 | 0.661 | 1.041 |
| 2-butene cis | 1.225 | 0.972 | 0.482 | 0.770 |
| 3-methyl butene-1 | 0.523 | 0.753 | 0.799 | 0.684 |
| Isopentane | 48.784 | 48.746 | 48.599 | 48.603 |
| Isopentene | 0.886 | 1.629 | 2.199 | 1.579 |
| 2-methyl butene-1 | 3.903 | 3.987 | 4.836 | 4.628 |
| Normal Pentane | 13.406 | 13.275 | 13.240 | 13.233 |
| Trans-2-pentene | 3.779 | 5.827 | 8.356 | 6.485 |
| Cis-2-pentene | 1.906 | 2.678 | 4.044 | 3.183 |
| 2-methyl butene-2 | 9.456 | 9.231 | 11.770 | 11.113 |
| Unknown C₁-C₅ | 0.003 | 0.004 | 0.002 | 0.003 |
| C₆⁺ | 10.127 | 7.777 | 3.180 | 5.875 |
| Total C5 = Conv | 42.641 | 32.399 | 10.427 | 22.396 |
| C4 = Selectivity | 25.475 | 26.346 | 22.627 | 24.888 |
| C6 + Selectivity | 66.605 | 67.317 | 87.034 | 73.567 |

### EXAMPLE IV

Table IV below shows data for gasoline which has been depentanized, the “Kettle Product.” The “Full Range” category denotes gasoline which also includes the C₅ components.

TABLE IV
Gasoline De-pentanization

| Gasoline Fraction | Full Range | Kettle Product |
|---|---|---|
| RON | 89.3 | 88.5 |
| MON | 80.1 | 79.1 |
| Rvp (psia @ 100° F.) | 4.82 | 2.27 |
| D-86 Data (° F.) | | |
| Initial Boiling Point | 115 | 156 |
| T10 | 162 | 191 |
| T50 | 255 | 268 |
| T90 | 388 | 389 |
| DI (calculated) | 1396 | 1479 |
| *DHA Results, vol % | | |
| C4 minus | 0.230 | 0 |
| C5 | 10.992 | 1.972 |
| C6+ | 88.778 | 98.028 |

Based on these data, the C5 fraction removed from gasoline has
blending RON of 95.8, blending MON of 88.2 and blending Rvp of
25.5; Measured C5 Rvp - 17.36 psig.
[*DHA = Detailed Hydrocarbon Analysis]

While this invention has been described in detail for the purpose of illustration, it should not be construed as limited thereby but intended to cover all changes and modifications within the spirit and scope thereof. Reasonable variations, modifications, and adaptations can be made within the scope of the disclosure and the appended claims without departing from the scope of this invention.

### REFERENCES

All of the references cited herein are expressly incorporated by reference. Incorporated references are listed again here for convenience:
1. U.S. Pat. No. 4,071,471 (Banks et al) “Catalysts for Conversion of Olefins”, granted Jan. 31, 1978.
2. U.S. Pat. No. 4,522,936 (Kubes et al) “Metathesis Catalyst”, granted Jan. 11, 1985.
3. U.S. Pat. No. 5,254,790 (Thomas et al) “Integrated Process for Producing Motor Fuels”, granted Oct. 19, 1993.
4. U.S. Pat. No. 6,395,945 (Randolph) “Integrated Hydroisomerization Alkylation Process”, grant May 28, 2002.

What is claimed is:

1. A process for upgrading hydrocarbons comprising:
a) separating a first hydrocarbon stream and a remaining hydrocarbon stream from a hydrocarbon feedstock in a first separation zone, wherein said first hydrocarbon stream comprises compounds having five carbon atoms per molecule (C5);
b) reacting said first hydrocarbon stream in a metathesis reaction zone to form a metathesis reaction product stream, wherein said metathesis reaction product stream comprises compounds having less than five carbon atoms per molecule (C4−), compounds having five carbon atoms per molecule (C5), and compounds having at least six carbon atoms per molecule (C₆₊);
c) separating a second hydrocarbon stream and a third hydrocarbon stream from said metathesis reaction product stream in a second separation zone, wherein said second hydrocarbon stream comprises compounds having less than six carbon atoms per molecule (C5−) and wherein said third hydrocarbon stream comprises compounds having at least six carbon atoms per molecule (C6+);
d) separating a fourth hydrocarbon stream and a fifth hydrocarbon stream from said second hydrocarbon stream in a third separation zone, wherein said fourth hydrocarbon stream comprising compounds having less than 5 carbon atoms per molecule (C4−) and said fifth hydrocarbon stream comprising compounds having 5 atoms per molecule (C5);
e) Reacting said fourth hydrocarbon stream in a hydrocarbon upgrading zone.

2. The process in according with claim 1 further comprising step selected from a list of steps consisting of i) passing said third hydrocarbon stream to a gasoline blending zone; ii) recycling said fifth hydrocarbon stream to said metathesis reaction zone; iii) passing said remaining hydrocarbon stream in first separation zone to said gasoline blending zone; and any combination thereof.

3. A process in accordance with claim 1 wherein said hydrocarbon upgrading zone is an alkylation reaction zone;

4. A process in accordance with claim 1 wherein said hydrocarbon upgrading zone is an oligomerization reaction zone.

5. A process in accordance with claim 1 wherein said metathesis reaction conditions include a reaction temperature in the range of from about 700° F. to about 800° F.

6. A process in accordance with claim 1 wherein said metathesis reaction conditions include a metathesis catalyst.

7. A process in accordance with claim 6 wherein said metathesis catalyst comprises silica, tungsten oxide, and magnesium oxide.

8. A process in accordance with claim 6 wherein said metathesis catalyst is regenerated with hydrogen.

9. A process in accordance with claim 1 wherein said hydrocarbon feedstock comprises compounds with 2 to 20 carbon atoms per molecule.

10. A process in accordance with claim 1 wherein said hydrocarbon feedstock contains less than 300 ppmw dienes.

11. A process in accordance with claim 1 wherein said hydrocarbon feedstock contains less than 100 ppmw dienes.

12. A process in accordance with claim 1 wherein said hydrocarbon feedstock contains less than 30 ppmw sulfur.

13. A process in accordance with claim 1 wherein said hydrocarbon feedstock contains less than 10 ppmw sulfur.

14. A process in accordance with claim 1 wherein said hydrocarbon feedstock contains less than 5 ppmw sulfur.

15. A hydrocarbon product stream prepared by the steps of:
a) separating a first hydrocarbon stream and a remaining hydrocarbon stream from a hydrocarbon feedstock in a first separation zone, wherein said first hydrocarbon stream comprises compounds having five carbon atoms per molecule (C5);
b) reacting said first hydrocarbon stream in a metathesis reaction zone to form a metathesis reaction product stream, wherein said metathesis reaction product stream comprises compounds having less than five carbon atoms per molecule (C4−), compounds having five carbon atoms per molecule (C5), and compounds having at least six carbon atoms per molecule (C₆₊);
c) separating a second hydrocarbon stream and a third hydrocarbon stream from said metathesis reaction product stream in a second separation zone, wherein said second hydrocarbon stream comprises compounds having less than six carbon atoms per molecule (C5−) and wherein said third hydrocarbon stream comprises compounds having at least six carbon atoms per molecule (C6+);
d) separating a fourth hydrocarbon stream and a fifth hydrocarbon stream from said second hydrocarbon stream in a third separation zone, wherein said fourth hydrocarbon stream comprising compounds having less than 5 carbon atoms per molecule (C4−) and said fifth hydrocarbon stream comprising compounds having 5 atoms per molecule;
e) Reacting said fourth hydrocarbon stream in a hydrocarbon upgrading zone.
